(19) United States
(12) Patent Application Publication
(10) Pub. No.: US 2011/0077292 A1
(43) Pub. Date: Mar. 31, 2011

(54) FORSKOLIN CARBONATES AND USES THEREOF

(75) Inventor: William J. Roberts, Ocala, FL (US)

(73) Assignee: BIOTEST LABORATORIES, LLC., Colorado Springs, CO (US)

(21) Appl. No.: 12/973,860

(22) Filed: Dec. 20, 2010

Related U.S. Application Data
(63) Continuation-in-part of application No. 12/626,717, filed on Nov. 27, 2009.
Application No. 12/626,717 is a continuation of application No. 10/883,995, filed on Jul. 1, 2004, now abandoned.

(51) Int. Cl. A61K 8/49; A61Q 90/00

(52) U.S. Cl. 514/452

(57) ABSTRACT

The present invention provides methods for reducing body weight. In particular, the present invention provides methods for reducing body weight by administering a composition comprising a forskolin 1α,9α-carbonate compound of the formula:

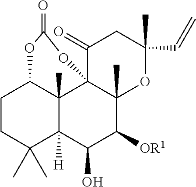

where R¹ is hydrogen or a carbonyl group.

### CROSS-REFERENCE TO RELATED APPLICATIONS

This application is continuation-in-part application of U.S. patent application Ser. No. 12/626,717, filed Nov. 27, 2009, which is a continuation of U.S. patent application Ser. No. 10/883,995, filed Jul. 1, 2004, abandoned, all of which are incorporated by reference in their entirety.

### FIELD OF THE INVENTION

The present invention relates to methods for reducing body weight. In particular, the present invention relates to methods for reducing body weight by administering a composition comprising a forskolin 1α,9α-carbonate compound.

### BACKGROUND OF THE INVENTION

Forskolin, sometimes referred to as forskolin, and also known as 8,13-epoxy-1α,6β,7β,9α-tetrahydroxylabd-14-en-11-one 7β-acetate, is a diterpenoid compound having the following chemical structure:

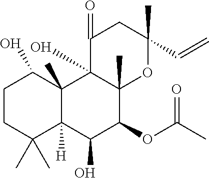

The numbering system for the carbon skeleton is well known to those in the art, and is disclosed, for example, in U.S. Pat. No. 4,088,659.

Forskolin is generally obtained by extraction from the plant known as coleus forskohlii. Traditional Ayurvedic medicine has used herbal preparations from coleus forskohlii for various therapeutic and medicinal purposes, including the treatment of heart and lung diseases, intestinal spasms, insomnia, convulsions, uterine cramps, painful urination, angina, and hypertension.

Forskolin is also reported to possess therapeutic and medicinal properties for treating many of the infirmities conventionally treated by coleus forskohlii. In addition, forskolin also reportedly has been used for other therapeutic purposes, such as ophthalmic (for lowering eye pressure and reducing the risk of glaucoma), asthma and other allergic conditions, and psoriasis, to name a few. Forskolin also has been proposed for clinical applications including weight-loss programs, hypothyroidism, malabsorption and digestive disorders, depression, prevention of cancer metastases, and immune system enhancement.

Without wishing to be bound by any theory, it is believed that forskolin activates the enzyme adenylate cyclase. Adenylate cyclase regulates the formation of cyclic adenosine monophosphate (cAMP), a compound that in turn exerts control over numerous cell activities. Once adenylate cyclase has been activated, the intracellular levels of cAMP are raised. Cyclic AMP activates many other enzymes involved in diverse cellular functions. Under normal circumstances, cAMP is formed when a stimulatory hormone, such as epinephrine, binds to a receptor site on the cell membrane and stimulates the activation of adenylate cyclase. Forskolin is believed to by-pass this need for direct hormonal activation of adenylate cyclase via transmembrane activation.

The physiological and biochemical effects of a raised intracellular cAMP level include inhibition of platelet activation and degranulation; inhibition of mast cell degranulation and histamine release; increased force of contraction of heart muscle; relaxation of arteries and other smooth muscles; increased insulin secretion; increased thyroid function; and increased lipolysis.

Recent studies have concluded that forskolin possesses additional mechanisms of action independent of its ability to directly stimulate adenylate cyclase and cAMP-dependent physiological responses. Specifically, forskolin has been reported to inhibit a number of membrane transport proteins and channel proteins through a mechanism that does not involve the production of cAMP. Another action of forskolin is on antagonizing the action of platelet-activating factor (PAF) by interfering with PAF binding to receptor sites. PAF plays a central role in many inflammatory and allergic processes, including neutrophil activation, increased vascular permeability, smooth muscle contraction including bronchoconstriction, and reduction in coronary blood flow.

Problematically, forskolin is believed to have relatively low bioavailability in human subjects when taken orally. Further, forskolin is believed to have an undesirably short duration of action, which may be only a few hours or less. In attempts to address these problems, numerous prodrugs of forskolin have been proposed in, for example, Sujata Bhat et al., The Antihypertensive and Positive Inotropic Diterpene Forskolin: Effects of Structural Modifications on its Activities, J. Med. Chem. 26, 486-492 (1983) (hereinafter “Bhat”), and J. Chem. So., Perkin Trans 1, 767 (1982). It is generally known in the art that in some instances, but not in others, prodrug derivitization of compounds may improve oral bioavailability and duration of action in human subjects. Prodrug derivitization is known in the art and referred to herein as the addition of one or more labile chemical groups (or promoieties) as substitute(s) for one or more atoms of the parent molecule. Bhat, however, has reported that forskolin prodrugs do not provide advantages over forskolin.

### SUMMARY OF THE INVENTION

Some aspects of the invention provide compositions and methods for reducing weight in a subject. Such methods include administering an effective amount of a composition comprising a forskolin 1α,9α-carbonate compound. The forskolin 1α,9α-carbonate compound of the invention is of the formula:

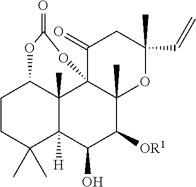

where R¹ is hydrogen or a carbonyl group. In some embodiments, R¹ is a carbonyl group. In other embodiments, R¹ is hydrogen or an acetyl group. Still in other embodiments, R¹ is hydrogen while in other embodiments R¹ is an acetyl group. In some embodiments, methods and compositions of the invention increase the in vivo concentration, bioavailability, and/or duration of action of forskolin derivatives disclosed herein.

Compositions of the invention can also include a lauroyl macrogol-32 glyceride.

In some embodiments, the composition is applied topically.

Other aspects of the invention provide methods for improving a skin condition in a subject comprising topically applying an effective amount of a skin conditioning composition to the subject. Typically, the skin conditioning composition comprises a forskolin 1α,9α-carbonate compound of the formula:

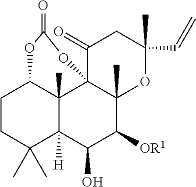

where R¹ is hydrogen or a carbonyl group.

In some embodiments, improving the skin condition comprises (a) beautification of skin, (b) strengthening of the epidermis, (c) promoting regeneration of the epidermis, (d) restoring elasticity, resilience or firmness of tissue, (e) improving overall color, texture and moisture retention of skin, (f) reducing creases or fine lines on the skin, or (g) a combination thereof. Compositions of the invention can also include a cosmetically acceptable excipient.

Still in other aspects of the invention, compounds of the invention are present in a cosmetic composition. Such cosmetic composition can be applied topically to promote fat loss or weight reduction.

Compositions of the invention can include a lauroyl macrogol-32 glyceride. Typically, a sufficient amount of the composition is administered to the subject such that the amount of forskolin 1α,9α-carbonate compound ranges from about 10 mg/day to about 80 mg/day, typically from about 20 mg/day to about 60 mg/day, and often about 30 mg/day. In some embodiments, the composition is administered to the subject twice daily, in which instances each of the two separate dosages comprises typically from about 20 mg to about 40 mg of the forskolin 1α,9α-carbonate compound.

The forskolin 1α,9α-carbonate compound can be made into a micronized form. It can be administered orally, but in many embodiments the composition is administered topically.

Some aspects of the invention provide a method for promoting fat loss in a subject by administering the composition of the invention.

### DETAILED DESCRIPTION OF THE INVENTION

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

In accordance with one aspect of the invention, a method is provided for the administration of a composition to a subject for reducing weight. The composition is typically administered to human. This is not necessarily limiting, however, and veterinary applications also are possible in certain instances. In some embodiments, the administration is to promote fat loss in the subject, and/or to reduce the weight in the subject, although other effects are possible.

Some methods of the invention include providing a composition that comprises a forskolin 1α,9α-carbonate (or “forskolin carbonate”) compound of the formula:

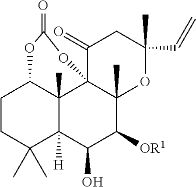

where R¹ is hydrogen or a carbonyl group. In some embodiments, R¹ is a carbonyl group. Exemplary carbonyl groups include formyl (—C(═O)H), acetyl (—C(═O)CH₃), etc. In other embodiments, R¹ is hydrogen or an acetyl group. Still in other embodiments, R¹ is hydrogen while in other embodiments R¹ is an acetyl group.

In accordance with another aspect of the invention, another method is provided for the administration of a composition to a human subject to promote fat loss.

In accordance with the methods pursuant to these aspects of the invention, the composition comprising the forskolin carbonate is administered to the subject. This administration is typically topical administration, although oral, transdermal administration and/or other means are also within the scope of the invention. The administration of the composition can also be by combinations of these techniques or approaches.

In some embodiments, the composition can include pharmaceutically acceptable excipients, such as powdered cellulose, and/or agents to facilitate bioabsorption and the like. The composition can also include a suitable carrier such as a lauroyl macrogol-32 glyceride, e.g., Gelucire 44/14 that is commercially available from Gattefosse Corporation.

The compositions comprising forskolin 1α,9α-carbonate compounds according to embodiments of the invention can be particularly useful, when applied topically, in aiding fat loss and/or reducing body weight of a subject.

The forskolin 1α,9α-carbonate compound of invention can be produced any of the suitable forms including: an ordinary or micronized powder; powder combined with cellulose and/or other pharmaceutically acceptable powdered excipient; in oil solution; in cosmetic formulation, etc.

Forskolin carbonate can also be provided in a self-emulsifying microemulsion formulation, for example, comprising a mixture of a cosurfactant with hydrophilic/lipophilic balance (HLB) of, e.g., approximately 5 or 6, and a surfactant with a HLB of approximately 14 or 15, in a weight/weight ratio of, for example, approximately 57/43. The solubility of forskolin carbonate in this formulation is typically at least 20 mg/mL. Typical co-surfactants are propylene glycol fatty acid esters with a hydrophilic/lipophilic balance of approximately 5 or 6, and more often propylene glycol caprylate esters. This also includes propylene glycol esters that are principally monoesters, for example, 60% or 90%. Examples include Capryol PGMC and/or Capryol 90, which are manufactured by Gattefosse Corporation. Another typically used co-surfactant is Plurol Oleique, also manufactured by Gattefosse Corporation. Plurol Oleique can also be used in a microemulsion formulation with another surfactant, or it can be used by itself or with an oil as an oily carrier without any other surfactant as excipient. A typical surfactant is polyoxyl caster oil, such as Cremophor EL and/or Cremophor RH, which are manufactured by BASF.

In some embodiments, compositions of the invention comprise forskolin carbonate in a self-emulsifying microemulsion formulation comprising about 50 weight percent of at least one member selected from Capryol PGMC, Capryol 90, and Plurol Oleique, and about 50 weight percent of at least one member selected from a polyethylene glycol-15-hydroxystearate, such as Solutol HS-15, manufactured by BASF. According to yet another embodiment, the forskolin carbonate is in a self-emulsifying microemulsion formulation comprising a lauroyl macrogol-32 glyceride, such as Gelucire 44/14.

Forskolin carbonate also can be provided in different self-emulsifying microemulsion formulations, for example, comprising a mixture of a surfactant with a hydrophilic/lipophilic balance (HLB) of approximately 10 or greater, typically 14 or 15, combined with a cosurfactant with a HLB of approximately 5 to 9, optionally with a lipoidal phase of HLB of 4 or less.

In some embodiments, the compositions are topical skin care compositions. The compositions can also be cosmetic compositions. In other aspects, the compositions can be included in a cosmetic vehicle. Suitable cosmetic vehicles are known to those skilled in the art. For example, suitable cosmetic vehicles include, but are not limited to, emulsions (e.g., oil-in-water and water-in-oil emulsions), creams, lotions, solutions (e.g., aqueous or hydro-alcoholic solutions), anhydrous bases (e.g., lipstick or a powder), gels, and ointments. In other embodiments, the compositions of the present invention can be included in anti-aging, skin-whitening/lightening, cleansing, or moisturizing products. Compositions can be oil-free, substantially anhydrous, and/or anhydrous.

The compositions of the present invention typically include from about 0.001% to about 50%, by weight, of the forskolin carbonate compound. However, it should be appreciated that the scope of the invention is not limited to such amounts and that the amount of forskolin carbonate can vary widely.

Compositions of the invention can optionally include a vitamin, a mineral, an essential fatty acid, an amino acid, a flavonoid, and/or a protein, or a combination thereof. Suitable examples of vitamins include the B vitamins (e.g., B₁, B₂, B₆, B₁₂, niacin, folic acid, biotin, and pantothenic acid), vitamin C, vitamin D, vitamin E (e.g., tocopherol or tocopheryl acetate), vitamin A (e.g., palmitate, retinyl palmitate, or retinoic acid), and vitamin K.

Acceptable examples of minerals include, but are not limited to, iron, potassium, phosphorus, magnesium, manganese, selenium, and calcium. Suitable essential fatty acids include, but are not limited to, Omega-3 fatty acids (e.g., linolenic acid), Omega-6 fatty acids (e.g., linoleic acid) and Omega-9 fatty acids (e.g., oleic acid), and other essential fatty acid, or a combination thereof. Exemplary amino acids that can be present in compositions of the invention include, but are not limited to, essential amino acids (e.g., lysine, leucine, isoleucine, methionine, phenylalanine, threonine, tryptophan, valine, histidine, or arginine) and non-essential amino acids (e.g., serine, asparagine, glutamine, aspartic acid, glutamic acid, alanine, tyrosine, cysteine, glycine, or proline). Suitable flavonoids include, but are not limited to, anthocyanin compounds (e.g., cyanidin-3-glucoside and cyanidin-3-rutinoside).

The cosmetic compositions of the invention can also include other ingredients such as a triglyceride, a preservative, an essential oil, a UV absorption ingredient, and/or additional ingredients described in the specification and known in the art, and any combination thereof. Such components are well known to those skilled in the art.

Other components that can be present in the compositions of the invention include water, butylene glycol, triethanolamine, and/or a preservative, dipotassium glycyrrhizate, ascorbyl glucoside, niacinamide, UV absorbing agents (e.g., homosalate, octisalate, oxybenzone, or avobenzone, or any combination thereof), glycerin, titanium dioxide, a biosaccharide gum, polyacrylamide, hydrolyzed jojoba esters, propylene glycol, glycerin, cyclopentasiloxane, alcohol, cetearyl ethylhexanoate, silica, glyceryl stearate, betaine, dimethicone, and/or cyclohexasiloxane, propylene glycol, dimethyl isosorbide, PEG-100 stearate, aluminum starch octenylsuccinate, a sunscreen agent, sodium cocoyl isethionate, stearic acid, cetyl alcohol, sodium methyl cocoyl taurate, cocamidopropyl betaine, PPG-5-Ceteth-20, or any combination thereof.

Other aspects of the invention provide methods of reducing weight or promoting fat loss comprising topical application of a composition comprising the forskolin carbonate compound.

Some aspects of the invention include methods for improving the skin condition by administering an effective amount of the composition of the invention. Often the composition of the invention is administered topically to improve the skin condition. Typical improvements in skin condition include (a) beautification of skin, (b) strengthening of the epidermis, (c) promoting regeneration of the epidermis, (d) restoring elasticity, resilience or firmness of tissue, (e) improving overall color, texture and moisture retention of skin, (f) reducing creases or fine lines on the skin, and/or (g) a combination thereof.

Methods of the invention can include topical application of the composition to a portion of skin in need of such composition (e.g., skin having a skin condition of dermatological or cosmetic nature), where topical application improves the skin condition or reduces the fat or weight when compared to skin that has a skin condition and that has not been treated with the composition. Exemplary skin conditions that can be treated by methods of the invention include, but not limited to, fat deposits, blotches, fine lines or wrinkles, dermatitis, etc.

The compositions and methods of the invention can be used for improving the skin's visual appearance, reducing weight or promoting fat loss in a particular area.

As discussed herein, topical skin care compositions of the invention include the forskolin carbonate compound. The forskolin carbonate compound can be used in relatively low amounts (e.g., the total amount of all ingredients within the formulation can range from 0.001 to 1% by weight, based on the total weight of the composition) and still produce the desired effects on the skin. However, it should be appreciated that the scope of the invention is not limited to these amounts of the forskolin carbonate compound. The amount of forskolin carbonate compound in a particular composition can be much higher to achieve a desired effect.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Example

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Synthesis of Forskolin Carbonate

To a flask fitted with a magnetic stirrer, forskolin (25 g, 63 mmol) and anhydrous pyridine were added under argon atmosphere. The mixture was stirred at 0° C., and phosgene (20% solution in toluene, 50 mL, 100 mmol) was added dropwise for 30 min. The mixture was stirred at room temperature for 4 hours, cooled to 0° C., and water (100 mL) was added dropwise. The resulting mixture was diluted with water (100 mL) and extracted with ethyl acetate (100 mL). The organic phase was separated, washed with 5% aq HCl (2.times.100 mL), and additional ethyl acetate (300 mL) was added. The solution was washed with 5% NaCl (400 mL), saturated NaCl (200 mL) and dried over anhydrous Na₂SO₄. The solution was concentrated under reduced pressure to give bulk crystallization of the product, and hexane (200 mL) was slowly added. The mixture was stirred for 3 h, the product was collected by filtration, and dried in vacuum. This yielded 22.7 g (86%) of forskolin carbonate material.

### Composition Formulation

It will be appreciated that topical application of forskolin carbonate for cosmetic or fat loss purposes is typically dependent not only on the properties of forskolin carbonate itself but also on the formulation. Certain formulations will be ineffective for any of various reasons, which may include inadequate amount of forskolin carbonate, low chemical potential of forskolin carbonate in the formulation, poor retention of formulation on the skin, or poor chemical stability of the formulation. Furthermore, formulations may be undesirable for reasons such as undesirable feel on the skin or adverse effect of the vehicle or other components on the skin.

Additionally, it will be appreciated that certain formulations have unusual benefit arising from high efficiency of delivery, sufficient amount of forskolin carbonate, sufficient chemical potential of forskolin carbonate in the vehicle, high retention of formulation on the skin, and good chemical stability of the formulation. Certain formulations may be particularly desirable for reasons of including the above advantages together with desirable feel on the skin or even, additionally, cosmetically or dermatologically advantageous properties of the vehicle and/or other included actives.

The chemical potential of a compound in a vehicle is understood by those skilled in the art to be at the highest stable value where a compound is supplied as a suspension or a saturated solution, and to be at proportionally lower values where the vehicle is only partially saturated with the compound. Additionally in some cases the chemical potential may be unusually high where loss of vehicle, e.g. by evaporation or absorption into the skin, results in a supersaturated state of the compound or in some cases an amorphous state with lower melting point than the usual crystal state.

The inventor has previously taught (see, for example, Roberts and Sloan, J. Pharm. Sci., 88 (1999) 515-522; Roberts and Sloan, J. Pharm. Sci., 89, (2000) 1415-1431; and Roberts and Sloan, Int. J. Pharm., 205 (2000) 53-63) that transdermal delivery and dermal delivery of a compound are functions of its lipid solubility, its aqueous solubility, and its percent saturation in the vehicle, with a suspension giving equal rate of delivery to a saturated solution. Forskolin carbonate has a superior combination of aqueous and lipid solubilities to forskolin itself and therefore for any percent saturation or for a suspension forskolin carbonate yields transdermal and superior dermal delivery superior to those of forskolin, an unanticipated result.

Those skilled in the art will understand that the examples provided are illustrative and not only can be used as given, but with ordinary experimentation can be adapted to specific commercial needs with no further invention required.

Examples of advantageous and effective formulations include but are not limited to:

### Formulation 1

Principal active:
Forskolin carbonate: 1200 mg

Additional optional ingredients, Group A, in any combination:
Sopholiance (available from Soliance Company): 2.0 mL
Theophylline: 3.2 grams
Yohimbine HCl: 180 mg

Typically all three of the optional ingredients in Group A are included. The most efficacious formulation will include all three, but cost advantages may be realized by omitting one or more of these components.

Additional optional ingredients, Group B, in any combination:
Glycerol: 1.0 mL
Sodium 2-pyrrolidone-5-carboxylic acid, 50% aqueous solution: 8.0 grams
Menthol 1.0 gram

The optional ingredients in Group B can be included or omitted according to a desired property. The first two components add moisturizing effect, while menthol increases transdermal delivery, adds a bracing feel to the formulation, and provides a distinctive scent.

Optional stabilizers:
Vitamin C: 500 mg
Vitamin C palmitate: 1000 mg
BHT: 200 mg
   Often all three of the optional stabilizers are included

Vehicle:
Transcutol (available from Gattefosse Corp.) 22 mL
Fragrance: As desired
Isopropyl alcohol, 91 to 100%: To full balance of volume.

This formulation is prepared by simple mixing of all ingredients. Application is to desired areas of the skin at an amount of approximately 1/30 mL per square inch. Total application should not exceed 5 mL at a time or more than 10 mL per day. The application may be performed by means of spraying or a sponge-type or roll-on applicator.

### Formulation 2

Active:
Forskolin carbonate, micronized: 1200 mg

Vehicle:
Mineral oil: 24 mL
Ethanol, 190 proof: 8.0 mL
Isopropyl palmitate: 1.2 mL
Fragrance: As desired
Masil SF V (available from Mazer Chemicals, Inc.): To total volume of 80 mL.

This formulation is typically prepared by mixing of all ingredients. Application is to desired areas of the skin at an amount of approximately 1/100 mL per square inch. Total application should not exceed 2 mL at a time or more than 4 mL per day. The application may be by hand or by an appliciance. This formulation is suited to applications where a skin oil is desired.

### Formulation 3

Active:
Forskolin carbonate, micronized: 1200 mg

Vehicle:
IPP (available from Unichema): 4.0 grams
Squalane (available from Polyesther): 8.0 grams
Cera Albalate 103 (available from Koster Keunen): 11.2 g
Apricot oil: 1.6 mL
Orange wax (available from Koster Keunen): 8.0 g
Vitamin A palmitate: 400 mg
Vitamin E: 400 mg
Candelilla wax (available from Koster Keunen): 1.6 g
Fragrance: As desired
Silicone oil 556: To total volume of 80 mL.

This formulation is typically prepared by mixing of all ingredients. Application is to desired areas of the skin at an amount of approximately 1/100 mL per square inch. Total application should not exceed 2 mL at a time or more than 4 mL per day. The application may be by hand or by an appliciance.

This formulation has anti-inflammatory, anti-oxidant, and anti-aging properties and is in the form of a deep-penetrating gel.

### Formulation 4

Active:
Forskolin carbonate, micronized: 1200 mg

Vehicle:
Arlacel 186 (available from ICI Americas): 36 mL
Arlamol E (available from ICI Americas): 36 mL
Cyclomethicone: 36 mL
Fragrance: As desired
Ethanol, 190 proof: To total volume of 120 mL.

This formulation is prepared by mixing of all ingredients. Application is to desired areas of the skin at an amount of approximately 1/50 mL per square inch. Total application should not exceed 4 mL at a time or more than 8 mL per day. The application may be by hand or by an appliciance, or may be via spraying.

The formulation is particularly suited to dry skin.

### Formulation 5

Active:
Forskolin carbonate, micronized: 1200 mg

Vehicle:
Liponate PC: 26 g
Crodamol PMP: 22 g
Liponate GC: 12 g
High oleic safflower oil: 3.6 g
Isoeicosane: 4.8 g
Ceraphyl 375: 3.6 g
Arlacel 186: 3.6 g
Propylparaben: 480 mg
BHT: 120 mg
Arlacel 186: 7.2 g
Fragrance: As desired
Isododecane: To total volume of 240 mL

This formulation is typically prepared by first mixing Arlacel 186 and any desired fragrance yielding Part A; then all other ingredients are mixed separately yielding Part B; and lastly Parts A and B are mixed together. Application is to desired areas of the skin at an amount of approximately 1/30 mL per square inch. Total application should not exceed 5 mL at a time or more than 10 mL per day. The application may be performed by means of spraying or a sponge-type or roll-on applicator.

The Liponate compounds are available from the Lipo Company. Crodamol PMP is available from Croda. The Arlacel compounds are available from ICI Americas. Isododecane and isoeicosane are available from Presperse.

This formulation is particularly desirable where spray application is required but alcohol content is undesired.

### Formulation 6

Active:
Forskolin carbonate, micronized: 1200 mg

Additional actives:
Hexaplant Richter: 2.4 g
Cosmedia Guar C-261: 0.8 g
Kathon CG: 16 mg
Nutrilan 1: 0.8 g

Vehicle:
Liponate PC: 13 g
Crodamol PMP: 11 g
Liponate GC: 6 g
High oleic safflower oil: 1.8 g
Isoeicosane: 2.4 g
Ceraphyl 375: 1.8 g
Arlacel 186: 1.8 g
Propylparaben: 240 mg
BHT: 60 mg
Arlacel 186: 3.6 g
Fragrance: As desired
Isododecane: To total volume of 120 mL.

The formulation is prepared by vigorous mixing of all ingredients. Application is to desired areas of the scalp via spraying in an amount causing light wetting of the scalp. Application may be once or twice per day, typically twice per day.

Hexaplant Richter, Cosmedia Guar C-261, Kathon CG, and Nutrilan 1 are available from Henkel Corp. Arlacel 186 and Arlamol E are available from ICI Americas.

This formulation is of particular value for stimulation of the scalp, including where stimulation of hair growth is desired.

### Formulation 7

Active:
Forskolin carbonate, micronized: 1200 mg

Vehicle:
Stearic acid: 2.4 g
C₁₂-C₁₅ Alkyl benzoate: 7.8 g
Cocoa butter: 1.2 g
Wheat germ oil: 1.2 g
Vitamin E acetate: 0.6 g
Glyceryl stearate: 2.4 g
PEG-40 stearate: 1.2 g
Aloe vera gel: 6.0 g
Glycerin: 3.6 g
Triethanolamine: 1.2 g
Allantoin: 0.6 g
Acritamer 941: 240 mg
Acrylates/Octylacrylamide copolymer: 1.2 g
Germaben IIE: 1.2 g
Defensine: 1.2 g
Tyrosine: 0-2.0 g
Methyl paraben: 0-300 mg
Fragrance: As desired
Arlacel 186: 20 mL
Arlamol E: 20 mL
Cyclomethicone: 20 mL
Isopropyl alcohol 91%: To total volume of 120 mL.

This formulation is prepared by mixing of all ingredients. Application is to desired areas of the skin at an amount of approximately 1/50 mL per square inch. Total application should not exceed 4 mL at a time or more than 8 mL per day. The application may be by hand or by an appliciance, or may be via spraying.

The formulation is particularly suited as a pre or after sun lotion.

Arlacel 186 and Arlamol E are available from ICI Americas. Germaben IIE and Defensine are available from RITA Corp.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for reducing the body weight of a subject comprising topically administering an effective amount of a composition comprising a forskolin 1α,9α-carbonate compound to reduce the body weight of the subject, wherein the forskolin 1α,9α-carbonate compound is of the formula: 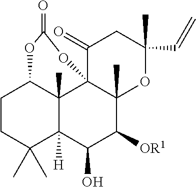
wherein R¹ is acetyl.

2. The method of claim 1, wherein the composition is a cosmetic composition.
